(19) United States
(12) Patent Application Publication
(10) Pub. No.: US 2015/0144144 A1
(43) Pub. Date: May 28, 2015

(54) Brain surgery through eye

(71) Applicant: Michael Lewis Moravitz, Fairfax, VA (US)

(72) Inventor: Michael Lewis Moravitz, Fairfax, VA (US)

(21) Appl. No.: 14/544,589

(22) Filed: Jan. 26, 2015

(51) Int. Cl. A61B 17/00

(52) U.S. Cl. CPC A61B 17/00; A61B 2017/00743
USPC 128/898

(57) ABSTRACT

Removing eye to remove malignant tumors in frontal lobe of brain.

Mortality from malignant brain tumors might be reduced from a procedure to remove an eyeball to obtain access to the tumor itself. This surgical procedure would result, of course, in the loss of the eyeball, but might save the patient's life.

The tumor would have to be located in the frontal lobe of the brain for the surgery to be considered. Then the eyeball would be carefully removed, and the surgeon would then remove the tumor. Clinical trials might be considered to determine if this new procedure would be successful, but given the low life expectancy of patients with malignant brain tumors, this surgery might become an option to chemotherapy. The patient then could wear a glass eye or eye-patch to cover the socket where the eyeball was removed.

1. Surgical procedures to remove eyes;

2. Surgical procedures to remove tumors from brain.
